(19) United States
(12) Patent Application Publication
(10) Pub. No.: US 2020/0346015 A1
(43) Pub. Date: Nov. 5, 2020

(54) NORMALIZATION OF BLOOD PRESSURE WITH SPINAL CORD EPIDURAL STIMULATION

(71) Applicant: UNIVERSITY OF LOUISVILLE RESEARCH FOUNDATION INC., Louisville, KY (US)

(72) Inventors: Susan J. Harkema, Louisville, KY (US); Claudia Angeli, Louisville, KY (US); Yangsheng Chen, Louisville, KY (US)

(21) Appl. No.: 16/960,447

(22) PCT Filed: Jan. 11, 2019
(86) PCT No.: PCT/US2019/013212

§ 371 Date: Jul. 7, 2020

Related U.S. Application Data
(60) Provisional application No. 62/616,714, filed on Jan. 12, 2018.

(51) Int. Cl. A61N 1/36; A61N 1/05

(52) U.S. Cl. CPC A61N 1/36117; A61N 1/36171; A61N 1/36175; A61N 1/36153; A61N 1/36062; A61N 1/0551; A61N 1/36139; A61N 1/36185

(57) ABSTRACT

Methods for normalization of blood pressure for individuals with spinal cord injuries include providing such individuals with spinal cord electrical stimulation optimized for cardiovascular function. An electrode array provides specific stimulation configurations identified to maintain systolic blood pressure within targeted normative ranges without skeletal muscle activity.

This application claims the benefit of U.S. provisional patent application Ser. No. 62/616,714, filed Jan. 12, 2018, for NORMALIZATION OF BLOOD PRESSURE WITH SPINAL CORD EPIDURAL STIMULATION, incorporated herein by reference.

### FIELD OF THE INVENTION

Methods for normalization of blood pressure for individuals with spinal cord injuries include providing such individuals with spinal cord electrical stimulation optimized for cardiovascular function. An electrode array provides specific stimulation configurations (anode and cathode electrode selection, voltage, frequency and pulse width) identified to maintain systolic blood pressure within targeted normative ranges without skeletal muscle activity.

### BACKGROUND OF THE INVENTION

Cardiovascular dysfunction is a leading cause of death in individuals with spinal cord injury and has a significant negative impact throughout their lifetime. Individuals with chronic spinal cord injury above T6 experience persistent hypotension and bradycardia, with orthostatic hypotension and severe increases in blood pressure during autonomic dysreflexia resulting in drastic daily fluctuations in cardiovascular activity. This dysregulation of the autonomic system affects their quality of life by causing discomfort, disrupting their ability to participate in rehabilitation, and interfering with their engagement in daily activities of life. Symptoms of chronic low blood pressure and orthostatic hypotension include fatigue, light-headedness, dizziness, blurred vision, dyspnea, and restlessness associated with cerebral hypo-perfusion.

The management of chronic hypotension in spinal cord injury is a challenging clinical issue as there is no standard or fully successful management strategy. Pharmacological interventions use agents that increase the sympathetic stimulation of the cardiovascular system or increase the blood volume in circulation. Undesirable side effects of pharmacological interventions and requirement of planning ahead of orthostatic stress have limited their application. Some non-pharmacological interventions have been used with limited success, which include applying external compression to reduce venous pooling in the abdomen or the lower extremities with the elastic compression stocking and abdominal binders. Other interventions to improve orthostatic hypotension include functional electrical stimulation, stand locomotor training, and respiratory motor training. Although these interventions obtained different degrees of success in reducing the severity of cardiovascular dysfunction, none have fully restored chronic hypotension.

### SUMMARY

In studies of motor behavior in individuals with motor complete spinal cord injury, we observed modulation of cardiovascular parameters when using spinal cord epidural stimulation (scES) at spinal segments L1-S1 (vertebrae T11-L1) that allows a direct modulation of the spinal neural networks below the level of injury. Targeted scES optimized for standing (Stand-scES) enabled four individuals with chronic motor complete spinal cord injury to achieve full weight-bearing standing with minimal assistance. Targeted scES optimized for voluntary movement (Vol-scES) enabled the same four individuals to perform intentional joint movements. These studies not only demonstrated effects of scES on altering the excitability of the spinal neural networks to process sensory and supraspinal inputs to improve motor behavior, but also highlighted the importance of optimization of the stimulation configurations, i.e., electrode set, frequency and intensity, to activate the neuronal pools to achieve targeted tasks and the stimulation parameters were also specific to individuals. During these studies we observed in two individuals who had orthostatic hypotension, blood pressure was elevated and symptoms of orthostatic hypotension were prevented during standing and stepping.

There have been animal studies and a proof of principle human study of neurally intact individuals showing that scES of the lumbar spinal cord has the potential to ameliorate chronic hypotension. Here, targeted scES optimized for solely cardiovascular function (CV-scES) provides a novel method of addressing cardiovascular dysfunction experienced in individuals with SCI. CV-scES at spinal cord segments L1-S1 with specific targeted configurations can increase resting blood pressure and attenuate orthostatic hypotension in individuals with chronic cervical SCI.

Chronic low blood pressure and orthostatic hypotension remain challenging clinical issues after severe spinal cord injury (SCI), affecting health, rehabilitation and quality of life. Here, CV-scES can increase resting blood pressure and attenuate chronic hypotension in individuals with chronic cervical SCI. Four research participants with chronic cervical SCI received an implant of a 16-electrode array on the dura (L1-S1 cord segments, T11-L1 vertebrae). Individual specific CV-scES configurations (anode and cathode electrode selection in the array, voltage, frequency and pulse width) were identified to maintain systolic blood pressure within targeted normative ranges without skeletal muscle activity of the lower extremities as assessed by electromyography. These individuals completed five two hour sessions using CV-scES in an upright, seated position during measurement of blood pressure and heart rate. Noninvasive continuous blood pressure was measured from a finger cuff by plethysmograph technique. For each research participant there were statistically significant increases in mean arterial pressure in response to CV-scES that was maintained within normative ranges. This result was reproducible over five sessions with concomitant decreases or no changes in heart rate using individual specific CV-scES that was modulated with modest amplitude changes throughout the session. These results indicate that stimulating dorsal lumbosacral spinal cord can effectively and safely activate mechanisms to elevate blood pressures to normal ranges from a chronic hypotensive state in humans with severe SCI with individual specific CV-scES.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein is not necessarily intended to address every object described herein, and each embodiment does not necessarily include each feature described. Other forms, embodiments, objects, advantages, benefits, features, and aspects of the present invention will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a fluoroscopy imaging chart showing electrode array located relative to thoracic and lumbar vertebrae. Large letters T11, T12, L1, L2 identify thoracic and lumbar vertebrae; smaller letters L1-S1 identify the spinal cord levels estimated by mapping with motor evoked potentials.

FIG. 2A includes a series of graphs depicting time courses of electromyography (EMG) and stimulation amplitude during 2 minutes of no stimulation (left), during 2 minutes of stimulation with configuration #1 at 30 Hz (middle) and stimulation with configuration #2 at 60 Hz (right) in one research participant B21. Electrode selection and stimulation frequency are represented on the top right of each section. In the depicted electrode arrays, grey boxes are cathodes, black boxes are anodes, and white boxes are inactive electrodes. EMG was recorded from the following muscles of the right lower limb: SOL, soleus; MG, medial gastrocnemius; TA, tibialis anterior; MH, medial hamstrings; VL, vastus lateralis; RF, rectus femoris.

FIG. 2B includes a series of graphs depicting amplitude of rectified EMG (left), systolic blood pressure (middle-left), diastolic blood pressure (middle-right) and heart rate (right) averaged over 2 minutes of no stimulation (white bars) and over 2 minutes of stimulation with configuration #1 (grey bars) and configuration #2 (black bars) from the data shown in (A). Error bars represent standard error of the samples of rectified EMG signal, and beat-to-beat values of systolic and diastolic blood pressure and heart rate.

FIG. 2C includes a series of graphs depicting time courses of EMG, electrocardiography (ECG) and continuous blood pressure (BP) in the 10-second windows entered in FIG. 2A.

FIG. 3 includes a pair of graphs depicting (A) systolic blood pressure and (B) heart rate responses to CV-scES in research participant A68 in the presence and absence of electrical stimulation. Systolic blood pressure gradually increased to target range and was maintained during 60 minutes of activation of stimulation (Stim ON) with minimal adjustment in stimulation amplitude, compared with baseline without stimulation (Stim OFF). Heart rate generally showed an inverse relationship with systolic blood pressure. Solid circles represent sitting systolic blood pressure (mmHg, top panel) and heart rate (beats per minute, bottom panel) averaged over every three minutes of beat-to-beat values (mean±SE). Grey shading area represents the target range of systolic blood pressure (110-120 mmHg). Stimulation (Stim) amplitude is shown in the middle and was continuous throughout the 60 minutes. Frequency was constant at 50 Hz. The vertical dash line indicates the start of stimulation. Electrode configuration is represented on the top right; grey boxes are cathodes, black boxes are anodes and white boxes are inactive electrodes.

FIG. 4A is a graph depicting mean arterial pressure response to CV-scES during participants' first session. Mean arterial pressure was significantly higher during CV-scES when compared with Pre-CV-scES and Post-CV-scES in all of the four research participants A41 (left panel), A68 (middle-left), B21 (middle-right) and A80 (right). Mean arterial pressure was significantly lower during Post-CV-scES compared to Pre-CV-scES in research participant A68 (middle-left) and higher in research participant A80 (right panel). Data points are one-minute averages of mean arterial pressure. Horizontal line: median; solid circle: mean; range of box: interquartile range (25th and 75th percentiles); whiskers: non-outliers maximum and minimum data points; open circles: outliers above or below 1.5× interquartile range. *: p<0.0001; #: p<0.003.

FIG. 4B is a graph depicting heart rate response to CV-scES during participants' first session. Heart rate was significantly lower during CV-scES compared to Pre-CV-scES in research participant A41 (left panel). Heart rate was significantly higher during Post-CV-scES when compared with Pre-CV-scES and CV-scES in all 4 research participants. Data points are one-minute averages of heart rate. Horizontal line: median;

solid circle: mean; range of box: interquartile range (25th and 75th percentiles); whiskers: non-outliers maximum and minimum data points; open circles: outliers above or below 1.5× interquartile range. Electrode configurations are represented on the bottom left of the bottom graphs, and apply to both FIG. 4A and 4B. Grey boxes are cathodes, black boxes are anodes and white boxes are inactive electrodes. *: p<0.0001; #: p<0.003.

FIG. 5 includes a pair of graphs depicting changes in (A) mean arterial pressure (delta mean arterial pressure) and (B) heart rate (delta heart rate) during the first thirty minutes of active CV-scES from Pre-CV-scES, averaged for four research participants for each of five sessions. Mean arterial pressure increased, and heart rate decreased, from Pre-CV-scES to CV-scES in all 5 sessions. Data points are one-minute averages of mean arterial pressure and heart rate subtracted from mean values of Pre-CV-scES. Horizontal line: median; solid circle: mean; range of box: interquartile range (25th and 75th percentiles); whiskers: non-outliers maximum and minimum data points; open circles: outliers above or below 1.5× interquartile range. Delta of mean arterial pressure and heart rate of all sessions were significantly different from zero (P<0.05).

FIG. 6 includes a series of eight graphs, designated A through H, depicting systolic blood pressure responses to optimal vs. non-optimal stimulation configurations in 4 research participants (A41: A and E; A68: B and F; B21: C and G; A80: D and H). Optimal stimulation configuration is important as non-optimal configurations failed to increase or maintain systolic blood pressure in the target range. Optimal stimulation configurations are shown in panels A, B, C, and D while non-optimal stimulation configurations are shown in panels E, F, G, and H. Solid circles represent systolic blood pressure averaged over every one minute of beat-to-beat values (mean±SE, left axis). Stimulation was continuous throughout the 10 minutes, black lines represent stimulation amplitude (right black axis) and grey lines represent stimulation frequency (right grey axis). Participant A80 was stimulated with three programs (D and H). In D and H, thin black line with squares represents stimulation amplitude of program #1 (P1), thick black line represents stimulation amplitude of program #2 (P2), thin black line with crosses represents stimulation amplitude of program #3 (P3). The locations of the squares and crosses in H indicate amplitude changes. Pulse width was 450 μs in all sessions and experiments for all participants. Grey shading areas represent the target range of systolic blood pressure (110-120 mmHg for research participants A41, A68 and B2; 105-115 mmHg for research participant A80). Electrode configurations are represented on the top right of each graph; grey boxes are cathodes, black boxes are anodes and white boxes are inactive electrodes.

### DETAILED DESCRIPTION OF THE PREFERRED EMBODIMENTS

Abbreviations and Acronyms:

| | |
|---|---|
| AIS | American spinal cord injury association |
| | impairment scale |
| CV-scES | spinal cord epidural stimulation |
| | targeted specifically for cardiovascular |
| | function |
| pre-CV-scES | Time period prior to beginning epidural |
| | stimulation targeted specifically for |
| | cardiovascular function |
| post-CV-scES | Time period following epidural |
| | stimulation targeted specifically for |
| | cardiovascular function |
| scES | spinal cord epidural stimulation |
| SCI | spinal cord injury |
| Stand-scES | standing targeted spinal cord epidural |
| | stimulation |
| Vol-scES | voluntary movement targeted spinal |
| | cord epidural stimulation |

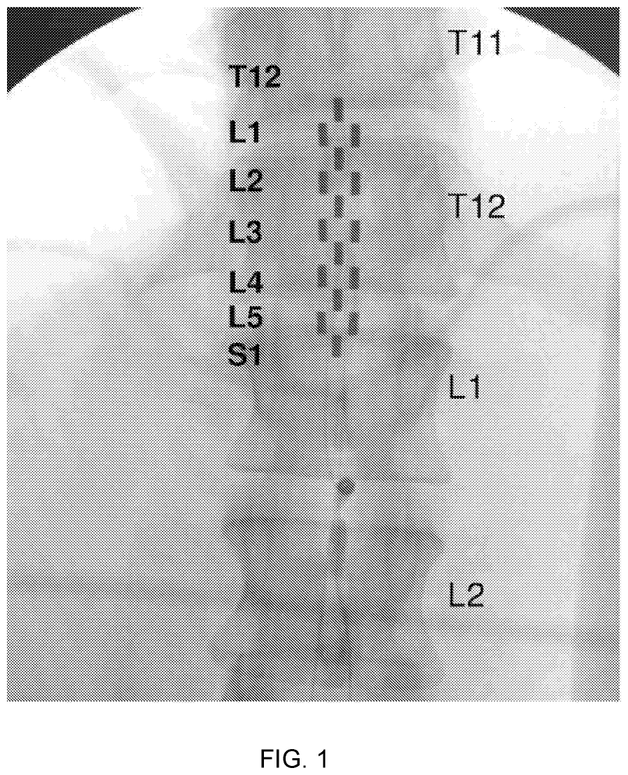

Four research participants with chronic motor complete, cervical SCI were studied (Table 1). All individuals were clinically stable, presented with orthostatic hypotension, persistent low resting blood pressure and routine symptoms of autonomic dysreflexia with no cardiovascular disease unrelated to SCI. A 16-electrode array (5-6-5 Specify, Medtronic) was implanted to span the spinal cord segments L1-S1 (vertebrae T11-L1) in each research participants (FIG. 1). The electrode lead was tunneled subcutaneously and connected to a pulse generator (RestoreADVANCED, Medtronic, Minneapolis, Minn., USA) placed ventral in the abdomen.

TABLE 1
Characteristics of SCI participants

| | | | Time | Neuro | | | |
|---|---|---|---|---|---|---|---|
| Partic- | | | since | Level | | | |
| ipant | | Age | injury | of | AIS | Weight | Height |
| ID | Gender | (years) | (years) | Injury | grade | (kg) | (cm) |
| A41 | Male | 24 | 7.2 | C4 | A | 84 | 185 |
| A68 | Male | 35 | 3.8 | C4 | A | 60 | 178 |
| B21 | Male | 31 | 7 | C4 | B | 86 | 183 |
| A80 | Female | 33 | 8 | C4 | A | 58 | 172 |

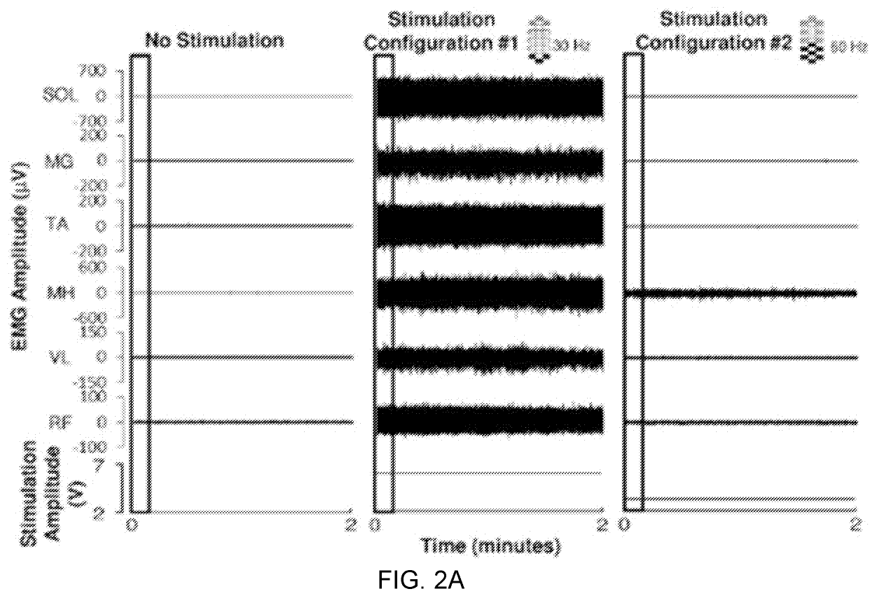

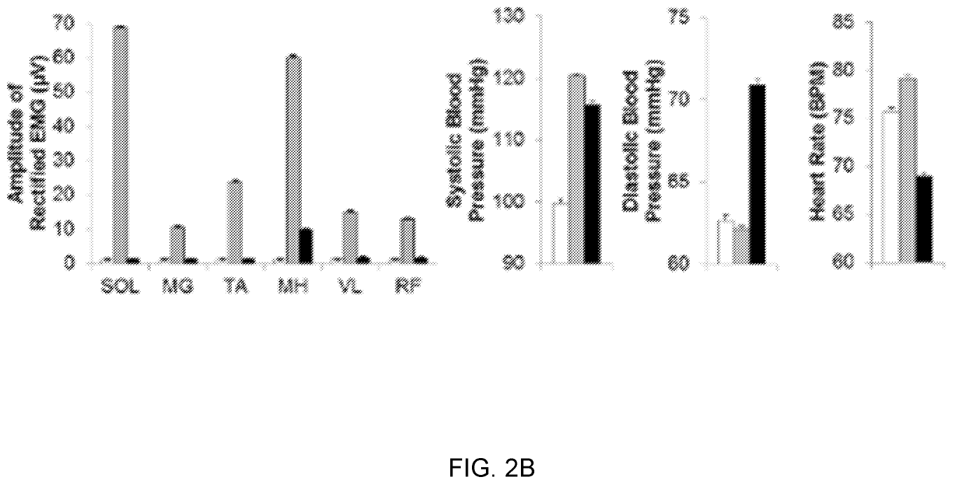

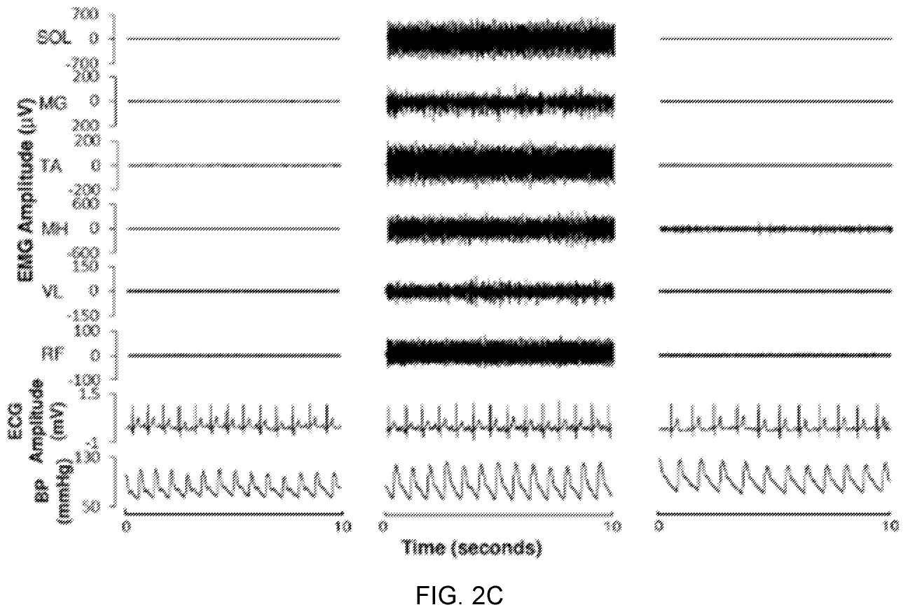

Research participants were resting in a seated position for two hours with continuous blood pressure and heart rate monitoring. CV-scES configurations (anode and cathode electrode selection, voltage, frequency and pulse width) were identified to maintain systolic blood pressure within the range of 110-120 mmHg for research participants A41, A68 and B21 and 105-115 mmHg for research participant A80, without skeletal muscle activity of the lower extremities as assessed by electromyography in mapping experiments. Exemplary mapping experiments for participant B21 are depicted in FIG. 2A, 2B, and 2C. Such mapping experiments typically required two to three sessions that were limited to two hours each. Then five two-hour stimulation sessions occurred where the CV-scES stimulation voltage and frequency would have been adjusted as needed throughout the session in order to target systolic blood pressure within the 110-120 mmHg range (for participants A41, A68 and B21) and 105-110 mmHg range (for participant A80). Pulse width was 450 μs in all sessions for all participants, although, in other embodiments, pulse width may be varied. Diastolic blood pressure and heart rate were also monitored to assure they were maintained within normative ranges. Resting data was obtained for 15 minutes before and after the two-hour stimulation period. Data from the first five cardiovascular sessions obtained within a two week period are presented herein.

Noninvasive continuous blood pressure measured from a finger cuff by plethysmographic technique (Finometer Pro, FMS, Amsterdam, Netherlands) was calibrated and recorded continuously in cardiovascular sessions. The continuous data were sampled with 1000 Hz sampling rate with an A/D converter device and computer interface (Powerlab 30/35 series and Labchart, AD Instruments, Colorado Springs, Colo., USA) and stored for offline analysis and was used to guide selection of initial stimulation parameters. Brachial blood pressure measured by oscillometric technique (Carescape V100, GE Healthcare, Milwaukee, Wis., USA) were obtained every 2-4 minutes during the session to calibrate finger blood pressure measurements during the experiments and post-analyses. During offline analysis, beat-to-beat systolic and diastolic blood pressure were calculated as the peaks and nadirs of the finger blood pressure waveform, after finger blood pressure was calibrated to brachial levels and internal calibration artifacts were removed. Mean arterial pressure was calculated as one third of systolic blood pressure plus two third of diastolic blood pressure. Heart rate was calculated from the interval between two beats. Beat-to-beat pressures and heart rate were then averaged for every 1 minute for statistical analysis. All analyses were performed with customized software in MATLAB (Mathworks, Natick, Mass., USA).

Data were summarized graphically using box plots. The box represents the interquartile range values (25th, 50th, 75th percentiles) and the whiskers extend to non-outliers minimum and maximum data points. Data points 1.5×IQR above the 75th percentile or below the 25th percentile were considered outliers. For each research participant and for the first session, three comparisons were performed for mean arterial pressure, systolic blood pressure, diastolic blood pressure and heart rate: CV-scES (during stimulation) vs pre-CV-scES (before stimulation); post-CV-scES (after stimulation) vs CV-scES (during stimulation) and post-CV-scES (after stimulation) vs pre-CV-scES (before stimulation). To perform these comparisons, linear mixed models on logged values were used including the experimental phase (pre-CV-scES, CV-scES, post-CV-scES) as fixed effect and the minutes as random effect nested within period phase, adjusted for the longitudinal nature of the data and the autoregressive nature of the data. Let y represent the outcome (mean arterial pressure, systolic or diastolic blood pressure or heart rate), the equation of the model was specified as follows: log (y_ij)=β_0+β_1* [symbol] time [symbol] _(i j)+β_2 [symbol] phase [symbol] _j+ε_ij with ε_ij=α_0+α_1*ε_(i−1,j) where i represents the time and j represents the experimental phase rank (pre-CV-scES=phase 1, CV-scES=phase 2 and post-CV-scES=phase 3). The three 2 by 2 comparisons were obtained by constructing linear contrasts from the model.

Each delta mean arterial pressure, delta systolic blood pressure and delta heart rate value during CV-scES was calculated as the difference of that value and the average of all pre-CV-scES values. These data were not normally distributed and they were also autoregressive (p-value of serial correlation <0.05). The signed rank test (comparing medians) adjusted for serial correlation using the method of effective sample size was used to test whether delta mean arterial pressure, delta systolic blood pressure, delta diastolic blood pressure and delta heart rate are different from 0. Statistical analyses were performed in SAS 9.4 (SAS Institute, Inc, Cary, N.C., USA).

Each individual completes three days of motor mapping of the electrode encompassing voltage response and frequency response curves of local two anode-cathode combinations rostral caudal and left right to determine the initial CV-scES configurations. EMG activity is recorded to identify those combinations that modulate blood pressure but do not elicit motor activity (FIGS. 2A, 2B, and 2C). EMG is at 2,000 Hz using a 24-channel hard-wired AD board and custom-written acquisition software (Labview, National Instruments, Austin, Tex.). EMG (MotionLab Systems, Baton Rouge, La.) from the soleus, medial gastrocnemius, tibialis anterior, medial hamstrings, rectus femoris and vastus lateralis using bipolar surface electrodes with fixed inter electrode distance. In addition, two surface electrodes were placed over the paraspinal muscles, symmetrically lateral to the epidural electrode array incision site. These two electrodes were used to record the stimulation artifact from the implanted electrode. EMG data were rectified and high-pass filtered at 40 Hz using Labview software customized by our laboratory. In these experiments we are very often modulating the parameters (anode, cathode selection; frequency and amplitude, number of programs) and the focus is to winnow down to successful CV-scES configurations. The goal is to find a CV-scES configuration that can maintain a relatively stable blood pressure within non-injured defined normal ranges for 2 hours without eliciting motor activity.

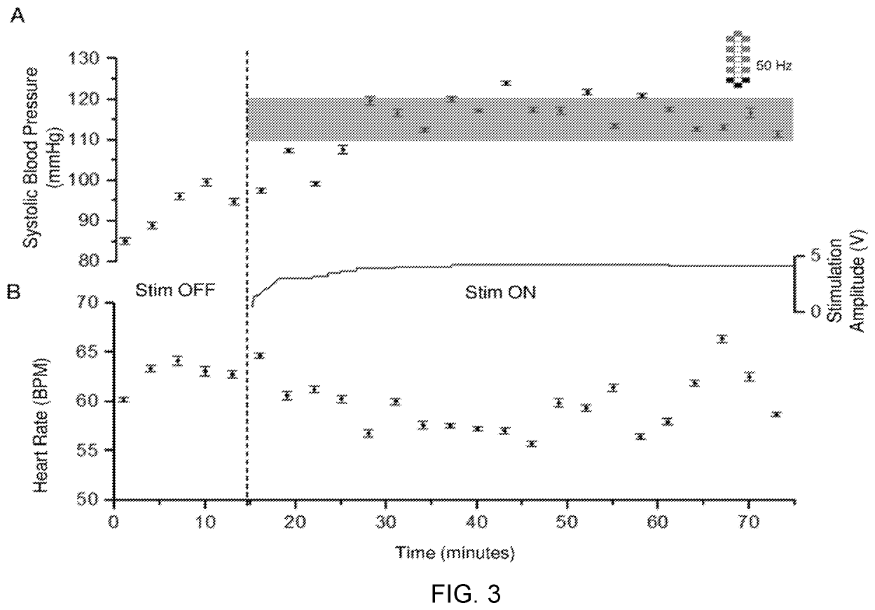

Cardiovascular parameters were normalized consistently in four individuals with chronic cervical spinal cord injury using participant specific CV-scES. Prior to stimulation there is often variability in the systolic blood pressure especially when below 90 mmHg as exemplified in participant A68 (FIG. 3). This typically occurs in response to yawning or involuntary muscle activation. Systolic blood pressure was gradually increased to the 110-120 mmHg or 105-115 mmHg range using the participant specific CV-scES configuration and could be maintained with minimal modulations in the stimulation amplitude. The stimulation amplitude was increased to increase systolic blood pressure and decreased to reduce systolic blood pressure to maintain the systolic blood pressure within the target range for 60 minutes. Generally, heart rate was inversely related to systolic blood pressure with some variability over time.

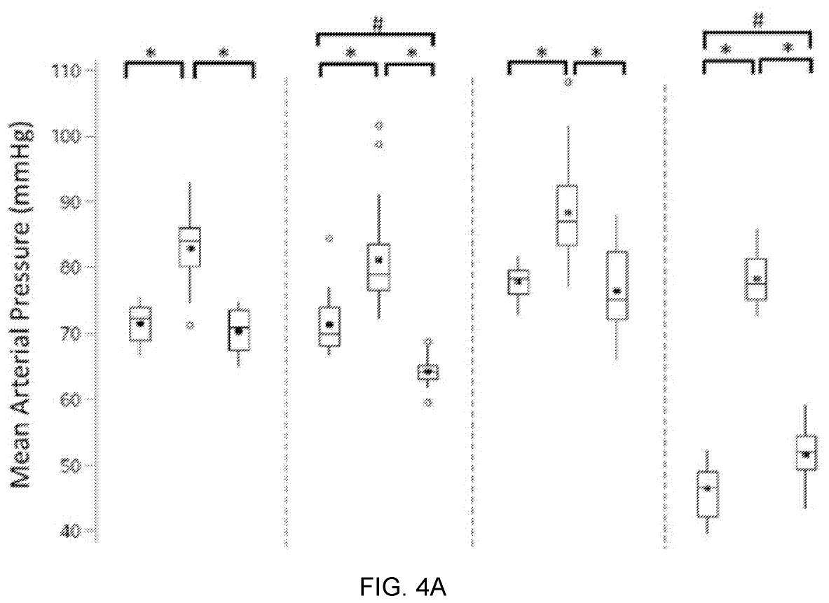

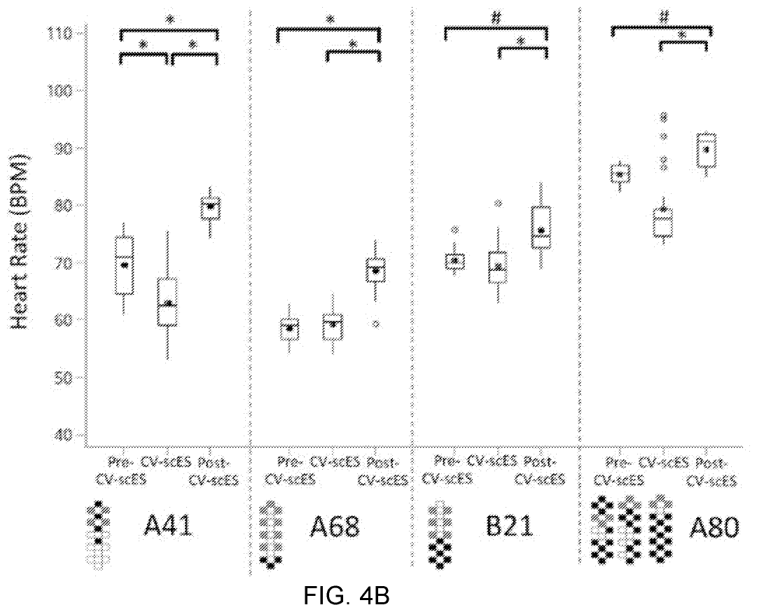

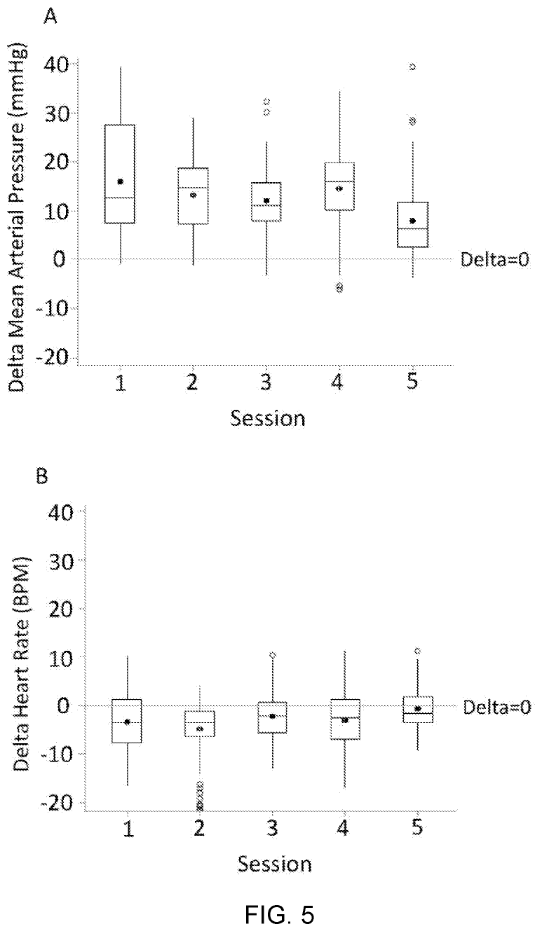

For each research participant there were statistically significant increases in mean arterial pressure in response to CV-scES (FIG. 4A). The mean arterial pressure changes represented concomitant increases in both systolic and diastolic blood pressures as detailed in Table 2. This rise in blood pressure returned near or below each participant's baseline values within 15 minutes of CV-scES cessation. Three individuals had no significant change in heart rate while A41 had a significant decrease during stimulation. After the CV-scES was turned off heart rate was significantly greater than the baseline for each participant (baseline shown in FIG. 3B, Stim OFF, data not shown for post-CV-scES heartrate). The average increases in mean arterial pressure during CV-scES for all four participants were reproducible as shown from five sessions over a two-week period with similar responses among the participants (FIG. 5A, Table 3). Individuals reported physical changes during these sessions including: 1) a feeling of alertness or heightened awareness; 2) increased ability to project their voice and carry on conversations; 3) increased capacity to breathe and cough; and 4) overall improved sense of well-being.

TABLE 2
Sitting systolic and diastolic blood pressure before and during
CV-scES at the first session day of each participant.

| | Systolic blood | | | Diastolic blood | | |
|---|---|---|---|---|---|---|
| | pressure, mmHg | | | pressure, mmHg | | |
| Partic- | Pre- | | | Pre- | | |
| ipant | CV- | CV- | P- | CV- | CV- | P- |
| ID | scES | scES | value | scES | scES | value |
| A41 | 101 ± 4 | 111 ± 5 | <.0001 | 57 ± 2 | 69 ± 5 | <.0001 |
| A68 | 97 ± 7 | 112 ± 11 | 0.0001 | 59 ± 4 | 66 ± 5 | <.0001 |
| B21 | 106 ± 5 | 118 ± 9 | <.0001 | 64 ± 2 | 74 ± 6 | 0.0002 |
| A80 | 65 ± 5 | 109 ± 8 | <.0001 | 37 ± 3 | 63 ± 3 | <.0001 |

Data presented as mean ± SD of minute-by-minute time series. CV-scES: cardiovascular targeted spinal cord epidural stimulation. Pre-CV-scES: before CV-scES. P-value: CV-scES versus Pre-CV-scES.

TABLE 3
Change in systolic and diastolic blood pressure during
CV-scES compared with baseline without CV-scES, combining
4 participants for each of the 5 session days.

| | Change in | | Change in | |
|---|---|---|---|---|
| | systolic | | diastolic | |
| Session | blood pressure, | P- | blood pressure, | P- |
| # | mmHg | value | mmHg | value |
| 1 | 20 ± 16 | 0.0418 | 14 ± 9 | 0.0221 |
| 2 | 18 ± 12 | 0.0184 | 11 ± 6 | 0.0026 |
| 3 | 14 ± 14 | 0.0004 | 10 ± 8 | <.0001 |
| 4 | 17 ± 10 | 0.0043 | 13 ± 7 | 0.0221 |
| 5 | 12 ± 11 | 0.008 | 6 ± 6 | 0.0031 |

Data presented as mean ± SD of minute-by-minute time series. CV-scES: cardiovascular targeted spinal cord epidural stimulation. P-value: change in systolic or diastolic blood pressure versus 0 mmHg.

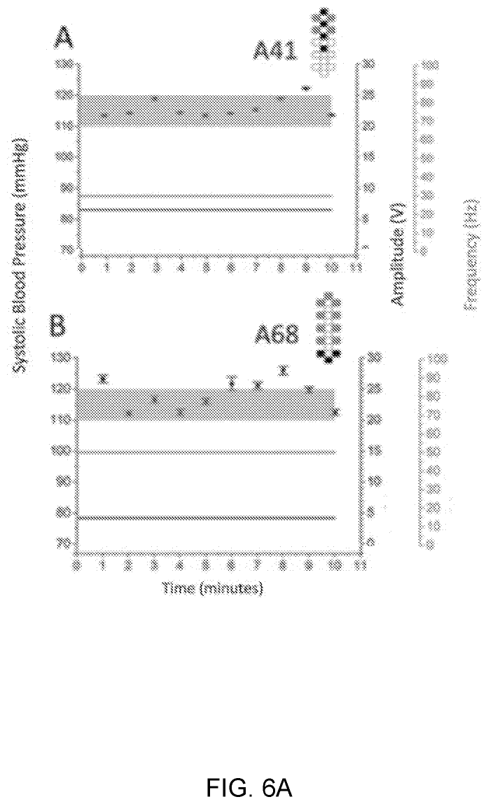

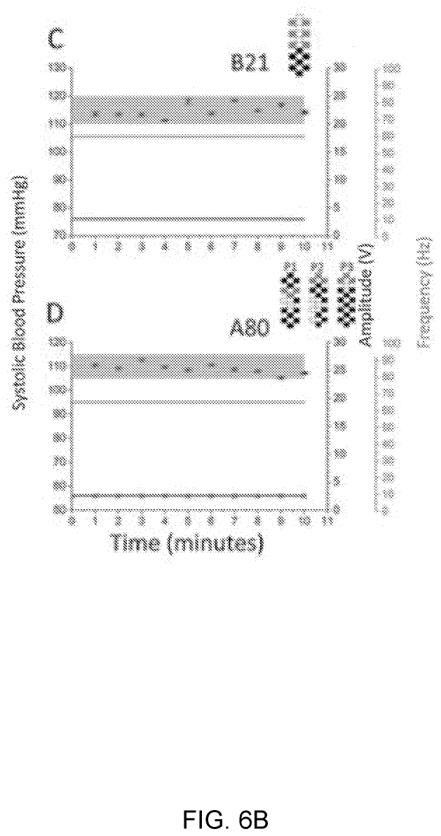

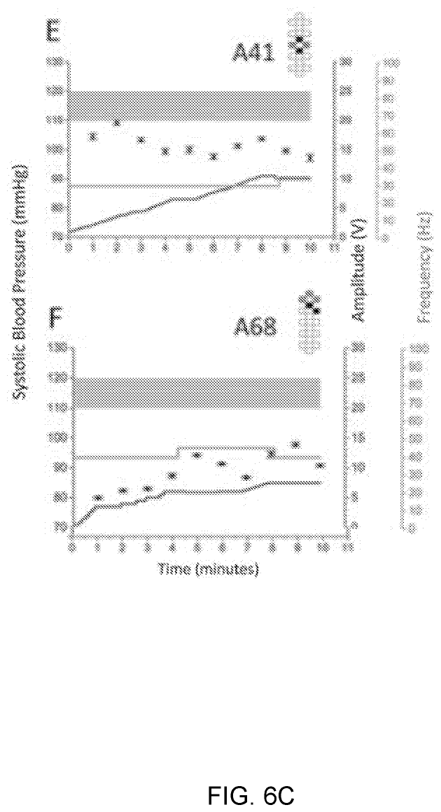

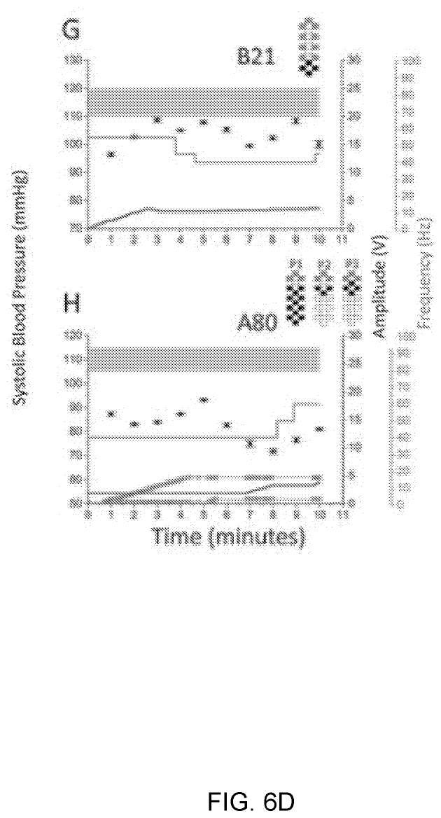

Each individual participant required a specific and unique CV-scES configuration (anode-cathode electrode selection, pulse width, frequency, amplitude) to consistently maintain normalized cardiovascular parameters. All four individuals had different configurations that maintained their systolic blood pressure stable within normal limits (FIGS. 6A-6D). In comparison, other CV-scES configurations not optimized for each individual were unable to consistently maintain blood pressure within the desired range of normalized parameters even when continually modulating stimulation amplitude (FIGS. 6E-6H).

Persistent hypotension was resolved in four individuals with chronic cervical SCI within normal blood pressure ranges using lumbosacral spinal cord epidural stimulation with customized targeted configurations for cardiovascular function. In all four participating individuals there were significant and reproducible increases in systolic and diastolic blood pressures (FIGS. 3A, 3B, 4A and 4B, Table 2). Heart rates decreased in one individual and remained relatively stable in the other three during stimulation. These individuals reported that they were invigorated, had better voice projection, were able to communicate more effectively, could breathe and cough better during these stimulation periods. The normotensive pressures were maintained for two hours with minor adjustments in amplitude or frequency and without activation of skeletal muscles. This effect was reproducible over multiple sessions (FIG. 5), and returned to low blood pressures when the stimulation ceased. As such, stimulating dorsal lumbosacral spinal cord can effectively and safely activate mechanisms to elevate blood pressures to a normal range from a chronic hypotensive state in humans with severe SCI with individual specific CV-scES for a controllable time period when the stimulation is present (FIG. 6).

Without being bound by theory, the eventual activation of sympathetic vasomotor efferents in the lumbosacral spinal cord causing vasoconstriction of the peripheral arteries leading to increase in venous return is a possible mechanism of the increase in blood pressure with CV-scES. The rise in blood pressure then likely stimulated the SCI participants' intact baroreceptors in the aortic arch and carotid sinus decreasing the heart rate via increased parasympathetic tone maintaining a stabilized system. For orthostatic hypotension or resting hypotension the lower thoracic levels for scES may also be efficacious in raising blood pressure due to the effects of efferent stimulation on the splanchnic vascular bed via sympathetic vasomotor efferents. It is conceivable that splanchnic vasoconstriction also could have contributed to venous return and increased blood pressure in response to CV-scES in the participating individuals with hypotension from secondary to chronic SCI, however it seems given that the site of stimulation is more consistent with the activation of the vasomotor sympathetic efferents from the lumbar cord being the more prominent mechanism responsible for the effect observed in this study given the similar location and type of response. However, other mechanisms must also be considered, for example, given the dorsal location of the electrode, dorsal fibers that project to intermedial lateral columns may reach and influence other levels of the spinal cord and aspects of the autonomic regulatory system.

This research found that to maintain normative blood pressures each person required unique CV-scES parameters and it took mapping for several hours using physiological measures to identify them. These studies concluded that spinal cord stimulation was safe, however there is significant variability with the site of stimulation, the pathophysiology, and the CV-scES parameters (electrode selection, etc.) among individuals. Each motor behavior and physiological response, such as blood pressure modification, has its own unique stimulation configuration indicating that the successful execution of the response is dependent on the appropriate excitability of the spinal network. Each individual also has a unique configuration and that may have many contributing factors including placement of electrode, differences among injuries, time since injury, pharmaceuticals and levels of activity.

The availability of implantable scES devices provides a novel treatment for chronic SCI patients to improve symptomatic resting and orthostatic hypotension. These findings are important not only to the chronic SCI population with substantial morbidity and mortality related to excess cardiovascular disease including extreme instability and liability of blood pressure control, but also to other disease states with significant morbidity from orthostatic hypotension such as multiple system atrophy (previously called the Shy-Drager syndrome) or baroreceptor-deafferentiated states such as occurs after carotid sinus disruption from trauma, vascular surgery or tumor involvement. Other dietary treatments such as salt loading or pharmacologic therapy with adrenergic agonists do not consistently prevent orthostatic hypotension and can lead to supine hypertension. CV-scES provides a novel treatment option for SCI patients to normalize blood pressure and alleviate the adverse impacts of chronic low blood pressure and orthostatic hypotension on physical, emotional and social well-being.

Various aspects of different embodiments of the present disclosure are expressed in paragraphs X1, X2, X3, and X4 as follows:

X1. One embodiment of the present disclosure includes a method for controlling blood pressure comprising providing an electrode array comprising a plurality of electrodes, wherein at least one of the plurality of electrodes is configured to deliver electrical stimulation to an individual; determining, for the individual, a configuration of electrical stimulation delivered by the electrode array effective in modifying a blood pressure of the individual; and delivering electrical stimulation to the individual, via the electrode array, according to the configuration.

X2. Another embodiment of the present disclosure includes a method of creating an electrical stimulation configuration for controlling an individual's blood pressure via electrical stimulation, the method comprising defining, for an individual having an implanted electrode array in operative engagement with the individual's spinal cord, a normalized blood pressure range and a baseline blood pressure, wherein the baseline blood pressure is outside the normalized blood pressure range; providing electrical stimulation to the individual using different combinations of electrodes in the electrode array; identifying at least one combination of electrodes which, when electrical stimulation is provided via the at least one combination of electrodes, modify the individual's blood pressure without producing a motor function in the individual; determining, for the identified combinations of electrodes, a frequency and amplitude of electrical stimulation effective in shifting the individual's blood pressure from the baseline blood pressure to the normalized blood pressure range; and defining the electrical stimulation configuration according to the at least one identified combinations of electrodes, the frequency, and the amplitude.

X3. A further embodiment of the present disclosure includes a method for increasing blood pressure in an individual, the method comprising delivering to the individual an effective spinal cord epidural stimulation.

X4. Another embodiment of the present disclosure includes a system for normalizing blood pressure in an individual with spinal cord injury, the system comprising an implantable electrode array, a pulse generator, and instructions for using the implantable electrode array and the pulse generator to normalize blood pressure.

Yet other embodiments include the features described in any of the previous paragraphs X1, X2, X3, or X4 as combined with one of more of the following aspects:

Wherein the configuration of electric stimulation includes a pattern of electrode activation, a frequency, a pulse width, and an amplitude.

Wherein the frequency, the pulse width, and the amplitude are independently controllable in each of the plurality of electrodes.

Wherein the individual has spinal cord injury.

Wherein the electrode array is in operative engagement with a portion of the individual's spinal cord.

Wherein the electrode array is implanted in operative engagement with thorasic and lumbar vertebrae in the individual's spinal cord.

Further comprising defining a normalized blood pressure range for the individual.

Wherein delivering includes decreasing an amplitude of the electrical stimulation when the individual's blood pressure exceeds the normalized blood pressure range.

Wherein delivering includes increasing an amplitude of the electrical stimulation when the individual's blood pressure is lower than the normalized blood pressure range.

Wherein the configuration of electrical stimulation does not produce a motor function in the individual.

Wherein the determining comprises activating combinations of electrodes to determine which modify the blood pressure of the individual without producing a motor function in the individual.

Wherein the individual is a human.

Wherein the individual has a spinal cord injury.

Wherein the spinal cord epidural stimulation is delivered by an implanted electrode array.

Wherein the spinal cord epidural stimulation is optimized for cardiovascular function.

Wherein the spinal cord epidural stimulation does not produce a motor function in the individual.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1. A method for controlling blood pressure comprising:
providing an electrode array comprising a plurality of electrodes, wherein at least one of the plurality of electrodes is configured to deliver electrical stimulation to an individual;
determining, for the individual, a configuration of electrical stimulation delivered by the electrode array effective in modifying a blood pressure of the individual; and
delivering electrical stimulation to the individual, via the electrode array, according to the configuration.

2. The method of claim 1, wherein the configuration of electric stimulation includes a pattern of electrode activation, a frequency, a pulse width, and an amplitude.

3. The method of claim 2, wherein the frequency, the pulse width, and the amplitude are independently controllable in each of the plurality of electrodes.

4. The method of claim 1, wherein the individual has spinal cord injury.

5. The method of claim 1, wherein the electrode array is in operative engagement with a portion of the individual's spinal cord.

6. The method of claim 1, wherein the electrode array is implanted in operative engagement with thorasic and lumbar vertebrae in the individual's spinal cord.

7. The method of claim 1, further comprising defining a normalized blood pressure range for the individual.

8. The method of claim 7, wherein delivering includes decreasing an amplitude of the electrical stimulation when the individual's blood pressure exceeds the normalized blood pressure range.

9. The method of claim 7, wherein delivering includes increasing an amplitude of the electrical stimulation when the individual's blood pressure is lower than the normalized blood pressure range.

10. The method of claim 1, wherein the configuration of electrical stimulation does not produce a motor function in the individual.

11. The method of claim 1, wherein the determining comprises activating combinations of electrodes to determine which modify the blood pressure of the individual without producing a motor function in the individual.

12. A method of creating an electrical stimulation configuration for controlling an individual's blood pressure via electrical stimulation, the method comprising:
defining, for an individual having an implanted electrode array in operative engagement with the individual's spinal cord, a normalized blood pressure range and a baseline blood pressure, wherein the baseline blood pressure is outside the normalized blood pressure range;
providing electrical stimulation to the individual using different combinations of electrodes in the electrode array;
identifying at least one combination of electrodes which, when electrical stimulation is provided via the at least one combination of electrodes, modify the individual's blood pressure without producing a motor function in the individual;
determining, for the identified combinations of electrodes, a frequency and amplitude of electrical stimulation effective in shifting the individual's blood pressure from the baseline blood pressure to the normalized blood pressure range; and
defining the electrical stimulation configuration according to the at least one identified combinations of electrodes, the frequency, and the amplitude.

13. A method for increasing blood pressure in an individual, the method comprising delivering to the individual an effective spinal cord epidural stimulation.

14. The method of claim 13, wherein the individual is a human.

15. The method of claim 13, wherein the individual has a spinal cord injury.

16. The method of claim 13, wherein the spinal cord epidural stimulation is delivered by an implanted electrode array.

17. The method of claim 13, wherein the spinal cord epidural stimulation is optimized for cardiovascular function.

18. The method of claim 13, wherein the spinal cord epidural stimulation does not produce a motor function in the individual.

19. The system for normalizing blood pressure in an individual with spinal cord injury, the system comprising an implantable electrode array, a pulse generator, and instructions for using the implantable electrode array and the pulse generator to normalize blood pressure.
